(19) United States
(12) Patent Application Publication
(10) Pub. No.: US 2026/0219228 A1
(43) Pub. Date: Jul. 30, 2026

(54) SENSING DEVICE HAVING PULSED REFERENCE ELECTRODES

(71) Applicant: AquaSpy, Inc., San Diego, CA (US)

(72) Inventors: Sotoudeh SEDAGHAT HOOR, Portland, OR (US); Peter ELLEGAARD, San Diego, CA (US)

(73) Assignee: AquaSpy, Inc., San Diego, CA (US)

(21) Appl. No.: 19/041,729

(22) Filed: Jan. 30, 2025

(51) Int. Cl. G01N 27/30; G01N 27/333; G01N 27/416; G01N 33/24

(52) U.S. Cl. CPC G01N 27/301; G01N 27/308; G01N 27/3335; G01N 27/4168; G01N 33/246

(57) ABSTRACT

Disclosed herein are reference electrodes, sensing devices including said reference electrodes, and methods of using the sensing devices to measure nitrate concentration of a surrounding medium. The sensing device includes at least one current source and at least one ionic sensor including a reference electrode configured to release and recapture ions based on current pulses received from the current source to generate a reference potential, a counter electrode configured to balance the release of ions from the reference electrode, and a nitrate-measuring electrode configured to detect electrochemical signals representative of the nitrate concentration of the surrounding medium based on the reference potential. The sensing device includes at least one controller electrically connected to the at least one ionic sensor to receive the electrochemical signals from the nitrate-measuring electrode and determine the nitrate concentration of the surrounding medium based on the electrochemical signals.

### FIELD

This application relates generally to sensing devices for measuring properties of a surrounding medium, and, more specifically, to sensing devices having ionic sensors that include pulsed reference electrodes for measuring properties of the surrounding medium.

### BACKGROUND

Nitrate in soil is produced as a result of biogeochemical processes involving moisture, soil organic matter, and microbial activity. It is a critical nutrient for plants; without it, crop yield and quality can suffer. Thus, nitrate can also be present in soil from ammonium-based fertilizers. Processes such as nitrogen mineralization, i.e., the conversion of organic nitrogen (e.g., from decaying plants, animals, and microorganisms) into ammonium, and nitrification, i.e., the biological oxidation of ammonium into nitrate by soil bacteria, produce nitrate. Moisture plays a key role in creating an environment conducive to these processes. For example, nitrification being an aerobic process requires oxygen to convert ammonium to nitrate. Thus, proper soil moisture ensures oxygen diffusion and microbial activity, in turn promoting efficient nitrification. Conditions that are too dry can reduce nitrification, while conditions that are too wet lead to waterlogged soil that creates anaerobic conditions and denitrification (i.e., reduction of nitrate back to nitrogen gas).

Excess water in the soil can also cause the nitrate ions to leach away from the root zone. This leaching can contaminate groundwater with the nitrate, which imposes serious health risks for humans. Nitrate leaching can also necessitate using additional fertilizers to maintain crop yield and quality, which increases production costs for farmers and the use of inorganic chemicals. Thus, both moisture and nitrate content of the soil must be taken into consideration together to achieve optimal crop irrigation practices.

Sensors for measuring soil properties such as ions (e.g., nitrate) in soil exist. These sensors include a reference electrode which generates an ion reservoir by continuously emitting a reference ion into the surrounding medium. The emitted ion forms an ionic reference potential against which the ion of interest is measured at the nearby working electrode. The problem with these sensors is that the emitted ion used to generate the ion reservoir, like the nitrate mentioned above, can leach into the soil over time. This becomes an issue when the sensors are buried deep into soil in the ground—the negative effects from leaching ions such as those described above with respect to nitrate can occur. Moreover, as the sensor continues to leach ions, the functionality of the sensor will deteriorate until it is rendered useless and requires replacement. Accordingly, a need remains for a sensor that can measure various soil properties, including ionic properties of the soil, over an extended period of time without necessitating replacement and while preventing ion leaching into the surrounding medium.

### SUMMARY

Described herein are sensing devices having pulsed reference electrodes for measuring ion concentration of a surrounding medium. The sensing device prevents ion leaching into the surrounding medium by being configured such that the reference electrode releases ions for measuring soil properties based on an applied current pulse and subsequently recaptures the ions following measurement based on another current pulse. By preventing ion leaching, the lifetime of the device is greatly extended and thus requires less frequent replacements once buried. Additionally, any negative effects associated with ion leaching into the surrounding medium such as soil are avoided.

The sensing devices described herein also improve irrigation practices in farming by providing greater intelligence regarding the state of the soil. Particularly, the sensing devices include an electrode for measuring ions such as nitrate, phosphate, and/or potassium. The sensing devices can also include an electrode for measuring the oxidation reduction potential (ORP) of the soil, and/or sensors for measuring moisture. In some examples, the sensing devices can also include an electrode for measuring pH, i.e., by detecting hydrogen ions within the soil. Together, the nitrate concentration, ORP, and moisture are interpreted holistically to determine optimal irrigation and fertilization parameters.

In some examples, a sensing device for measuring nitrate concentration of a surrounding medium is provided, the sensing device comprising: at least one current source; at least one ionic sensor comprising: a reference electrode configured to release and recapture ions based on current pulses received from the current source to generate a reference potential; a counter electrode configured to balance the release of ions from the reference electrode; and a nitrate-measuring electrode configured to detect electrochemical signals representative of the nitrate concentration of the surrounding medium based on the reference potential; and at least one controller electrically connected to the at least one ionic sensor to receive the electrochemical signals from the nitrate-measuring electrode and determine the nitrate concentration of the surrounding medium based on the electrochemical signals.

In some examples, a method for measuring nitrate concentration of soil is provided, comprising: generating a current pulse between a reference electrode and a counter electrode of a sensing device inserted into soil, whereby the first pulse of current causes release of ions from the reference electrode that generates a reference potential; and following the current pulse and based on the reference potential, measuring electrochemical signals representative of nitrate concentration of the soil between the reference electrode and a nitrate-measuring electrode of the sensing device.

In some examples, a reference electrode is provided, comprising: a copper layer; a gold layer disposed on the copper layer; a carbon layer comprising a first portion disposed on the gold layer and a second portion adjacent to the first portion; and a silver layer disposed on the second portion of the carbon layer; a coating disposed on the first portion of the carbon layer and the silver layer such that a portion of the silver layer is exposed; and a silver iodide layer electrodeposited in the exposed portion of the silver layer.

It will be appreciated that any of the variations, aspects, features and options described in view of the systems apply equally to the methods and vice versa. It will also be clear that any one or more of the above variations, aspects, features and options can be combined.

### BRIEF DESCRIPTION OF THE FIGURES

The invention will now be described, by way of example only, with reference to the accompanying drawings, in which:

FIG. 1 shows an exemplary sensing device for measuring properties of a surrounding medium, in accordance with some aspects.

FIG. 2 shows a schematic diagram of exemplary ionic sensors and moisture sensors of the sensing device, in accordance with some aspects.

FIG. 3 shows a schematic diagram of an exemplary sensing system, in accordance with some aspects.

FIG. 4 shows an exemplary ionic sensor assembly of the sensing device, in accordance with some aspects.

FIG. 5 shows a diagram of an exemplary reference electrode of an ionic sensor, in accordance with some aspects.

FIG. 6A shows an exemplary diagram of iodine ion release from the reference electrode and subsequent nitrate detection at the nitrate-measuring electrode; and FIG. 6B shows an exemplary diagram of iodine ion recapture to the reference electrode, in accordance with some aspects.

FIG. 7 shows a diagram of an exemplary activation circuit for driving the ionic sensor assembly, in accordance with some aspects.

FIG. 8 shows a diagram of an exemplary probe for sensing moisture of a surrounding medium, in accordance with some aspects.

FIG. 9A shows measured moisture data using an exemplary sensing device; FIG. 9B shows measured nitrate data using the exemplary sensing device; and FIG. 9C shows measured oxidation reduction potential (ORP) data using the exemplary sensing device, in accordance with some aspects.

### DETAILED DESCRIPTION

It will be appreciated that any of the variations, aspects, features and options described in view of the systems apply equally to the methods and vice versa. It will also be clear that any one or more of the above variations, aspects, features and options can be combined.

Described herein are sensing devices having pulsed reference electrodes for measuring various properties such as ion concentration of a surrounding medium. The sensing device minimizes ion leaching into a surrounding medium by delivering current pulses to the reference electrode that cause release and subsequent recapture of ions electrodeposited on the surface of the reference electrode. In turn, the longevity of the device is increased and requires less frequent replacements and/or calibration after being deposited in the surrounding medium. This is especially beneficial in the context of farming, where the sensing devices are oftentimes buried deep into the soil with sensors at varying depths. In this configuration, ionic sensors having reference electrodes can be especially susceptible to degradation from the soil that requires frequent replacement or recalibration of the device. The sensing devices described herein overcome this problem by providing a pulsed reference electrode configuration which extends the lifetime of the device by recapturing the ions emitted for measuring soil properties, thus preventing the ions from leaching into the soil.

The pulsed reference electrodes described herein are of ionic sensors for measuring several properties of the surrounding medium, which together provide greater intelligence as to the state of the surrounding medium. Aside from the pulsed reference electrode, the ionic sensors include an electrode for measuring ions such as nitrate, potassium, and/or phosphate (e.g., a nitrate-measuring electrode). In some examples, said electrode may measure pH by detecting hydrogen ions in the surrounding medium. In some examples, the ionic sensor can include an electrode for measuring oxidation reduction potential (ORP, e.g., an ORP-measuring electrode). The nitrate-measuring electrode measures electrochemical signals representative of nitrate concentration against the reference potential generated by the ion release at the reference electrode. The ORP-measuring electrode measures electrochemical signals representative of the ORP of the soil. The sensing device can further include one or more moisture sensors for measuring moisture content of the surrounding medium. Using this information collectively, the sensing devices described herein greatly improve irrigation practices in farming by enabling the user to make data-backed decisions regarding, for example, the optimal parameters for irrigation and fertilization (e.g., amount, cadence, duration, etc.). In turn, the biological oxidation of ammonium into nitrate is promoted, and the use of inorganic chemical fertilizers is reduced. Moreover, overwatering of the crops, which causes ions leaching away from the crop and into the groundwater, is prevented.

The following description details exemplary sensing devices having ionic sensors that include pulsed reference electrodes. The release and recapture of ions from the reference electrode for measuring properties by the ionic sensor is described. Also, the actuation of the pulsing of the reference electrodes is also described. Additionally, the exemplary sensing devices are further described with regards to the moisture sensors that may be included therein. Finally, exemplary data captured using the sensing devices described herein is described.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

### Sensing Devices & Systems

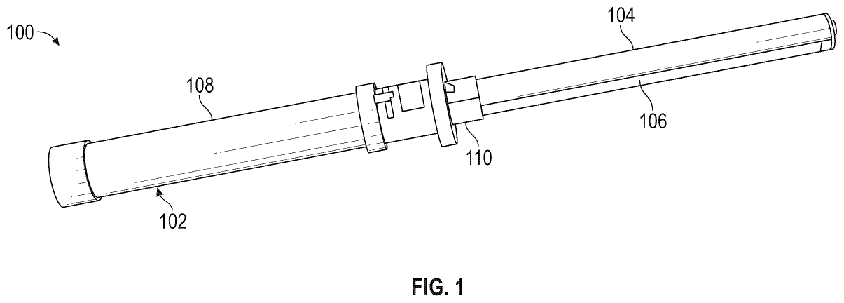

FIG. 1 shows an exemplary sensing device 100 for measuring properties of a surrounding medium, in accordance with some aspects. As depicted in FIG. 1, the sensing device 100 may be in the form of an elongated housing 102, although the sensing device 100 is not intended to be limited to this and may take another form. The housing 102 may be composed of a non-metallic material such as polyvinyl chloride (PVC) or another similar material.

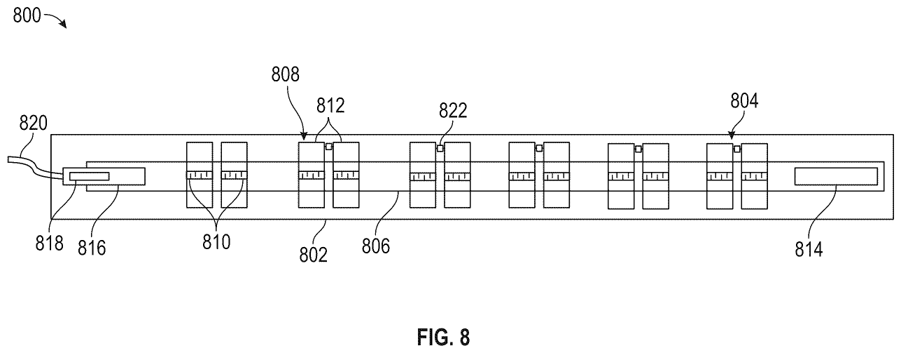

The housing 102 can include a first portion and a second portion. The first portion can be a sensor housing portion 104 that supports one or more sensor assemblies for measuring properties of the surrounding medium. For example, the sensor housing portion 104 can support an ionic sensor assembly 106 for measuring ion (e.g., nitrate) concentration. In some examples, the ionic sensor assembly 106 can also measure oxidation reduction potential (ORP) of the surrounding medium. The ionic sensor assembly 106 may be positioned on an external surface of the sensor housing portion 104 of the housing 102 to measure ion concentration, such as nitrate, in the surrounding medium. The sensor housing portion 104 can support a probe for sensing moisture that is contained within the sensor housing portion 104 (illustrated and described in greater detail below with respect to FIG. 8).

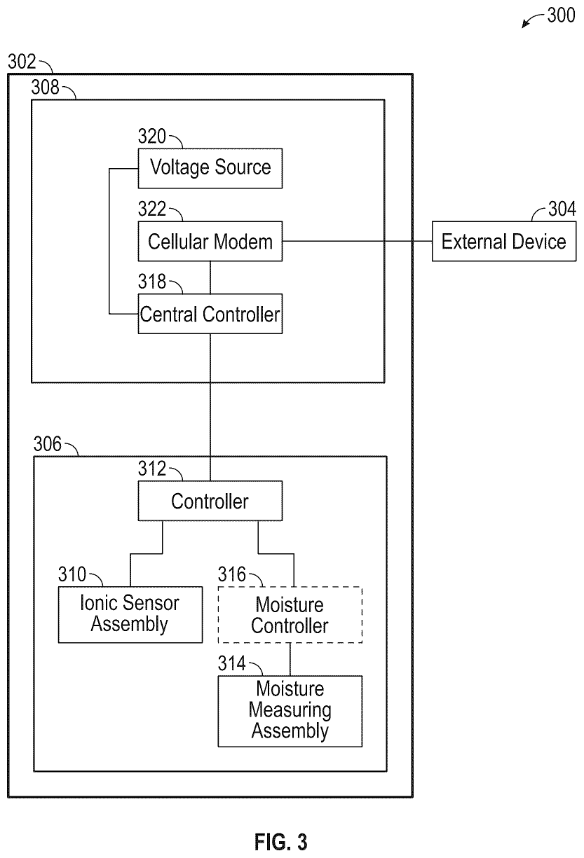

The second portion of the housing 102 can be a telemetry housing portion 108 that supports the telemetry components of the device, as described in greater detail with respect to FIG. 3. The sensor housing portion 104 and telemetry housing portion 108 can be removably attachable at a connector 110. By being removably attachable, either of the sensor housing portion 104 and telemetry housing portion 108 of the housing 102 (or components supported thereby) may be replaced rather than replacing the whole sensing device 100.

At least a portion of the sensing device 100 can be inserted to a desired medium for measuring properties of the medium. For example, at least the section of the sensor housing portion 104 supporting the sensing assemblies may be inserted to the surrounding medium for proper measurement of the properties of the surrounding medium. The surrounding medium may be soil.

In some examples, a portion of the sensing device 100, such as a section of the telemetry housing portion 108, may remain outside of the surrounding medium for visibility of the sensing device 100 relative to the surrounding medium. Keeping at least a section of the telemetry housing portion 108 outside of the surrounding medium can also enable transmission of signals between telemetry components supported by the second portion 108 and an external device, as described in greater detail below with respect to FIG. 3.

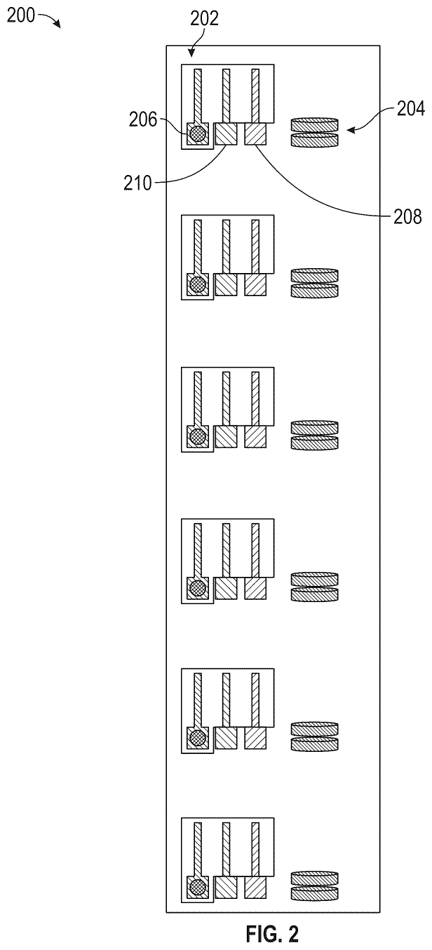

FIG. 2 shows a schematic diagram of a sensing device 200 that can be used for the sensing device 100. The sensing device 200 can include one or more ionic sensors 202 and one or more moisture sensors 204. For example, the sensing device 200 may include at least 1, 2, 3, 4, 5, 6, 7, 8, or more the ionic sensors 202 and moisture sensors 204. The number of each of the sensors may be dependent on the size (e.g., length) of the sensing device 200.

Each ionic sensor 202 can include a reference electrode 206, a working (e.g., nitrate-measuring) electrode 208, and a counter electrode 210. Unless stated otherwise herein, it is to be understood that the working electrode may be configured to measure an ion different from nitrate in the surrounding medium and is not intended to be limited to this ion. For example, the working electrode 208 may be configured to measure potassium, phosphate, hydrogen (corresponding to pH), etc. The counter electrode 210 may be configured to measure the oxidation reduction potential (ORP) of the surrounding medium and may otherwise be referred to herein as an ORP-measuring electrode. As will be discussed in greater detail below, the reference electrode 206 may couple to at least one current source to selectively release ions to the surrounding medium, in turn creating a known potential. The working electrode 208 can detect a potential difference (i.e., electrochemical signals) based on the known potential of the reference electrode and interaction of the nitrate ions with the working electrode 208. The measured electrochemical signals can be proportional to the nitrate concentration in the surrounding medium. The counter electrode 208 can detect electrochemical signals representative of the oxidation-reduction potential (ORP) between the reference electrode 206 and the working electrode 208. In the context of farming, the ORP can inform whether the soil is in a nitrification or denitrification phase, i.e., whether nitrate is being naturally generated or reduced in the soil.

Each ionic sensor 202 may be positioned proximate to a moisture sensor 204. For example, the ionic sensor 202 and the moisture sensor 204 may be positioned such that each of the sensors can measure properties of the surrounding medium at about the same depth in the surrounding medium. The moisture data measured from the moisture sensor 204 can be used to validate the data generated by the ionic sensor 202. This is because the ionic sensor 202 (i.e., the reference electrode 206, working electrode 208, and counter electrode 210), forming an electrogalvanic cell, may be functional only when enough liquid (moisture) exists in the medium. The ionic sensor potential may remain stable until the medium reaches a lower threshold of moisture, at which point the potential field may change significantly. Thus, the moisture data can ensure accuracy of the measurements from the ionic sensor 202.

FIG. 3 shows a schematic diagram of an exemplary sensing system 300, in accordance with some aspects. The sensing system 300 can include a sensing device 302 and an external device 304 communicatively coupled (e.g., via a wireless connection) to the sensing device 302. The sensing device 302 is understood to be representative of the sensing devices described herein, e.g., sensing device 100. For example, the sensing device 302 can include a sensor housing portion 306 and a telemetry housing portion 308, as noted above. The sensing housing portion 306 may support the sensing components, e.g., the ionic sensor assembly 310 and the moisture sensor assembly 314. The telemetry housing portion 308 may support telemetry components that communicatively couple to the external device 304. As explained herein, the sensing housing portion 306 and the telemetry housing portion 308 may be removably attachable. In some examples, the sensing device 302 includes a single housing that includes both the telemetry and sensing components.

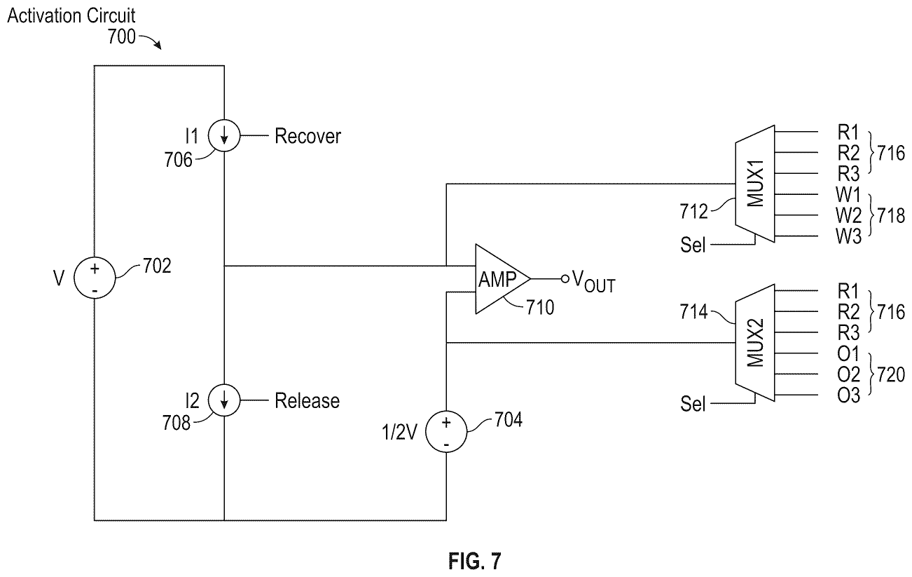

The sensing device 302 can include one or more controllers communicatively coupled to the sensor assemblies for activating the sensors and/or receiving measured data from the sensors. For example, the sensing device 302 can include a controller 312 communicatively coupled to each of the ionic sensor assembly 310 and the moisture measuring assembly 314. The controller 312 may selectively activate sensors of the ionic sensor assembly 310 and/or of the moisture measuring assembly 314. For example, as described in greater detail below with reference to FIG. 7, the controller 314 may include one or more multiplexers for selectively driving current pulses from at least one current source to the ionic sensors of the ionic sensor assembly 310. The controller 312 may receive electrochemical signals detected by the sensors of the ionic sensor assembly 310 and/or moisture measuring assembly 314. In some examples, the controller 312 may process the received signals to determine a corresponding property (e.g., ion concentration, moisture content, ORP).

One or more of the functions of the controller 312 may be delegated to other controllers in the system. For example, the sensing device 302 may optionally include a separate controller 316 for controlling and/or receiving data from the moisture measuring assembly 314. In this example, the controller 316 may be communicatively coupled to the controller 312, for example, to transmit data measured by the moisture measuring assembly 314 to the controller 312. In some examples, the central controller 318 may process the measured data received at the controller 312.

The sensing device 302 can include at least one voltage source 320, such as at least one battery, electrically coupled to the central controller 318. The voltage source 320 may power the various controllers (and, in turn, the sensing assemblies). In some examples, the at least one voltage source 320 is rechargeable (e.g., via the cellular modem 322).

In some examples, measured data (e.g., ion concentration data, ORP data, and/or moisture content data) may be transmitted to the external device 304 via the cellular modem 322 for further processing. In some examples, measured data may be transmitted to the external device 304 for storage on the external device 304 or another medium associated with the external device 304. In some examples, measured data may be transmitted to the external device 304 for display and analysis of the measured data.

In some examples, parameters for operating the sensing assemblies may be set at one of the controllers. In some examples, these parameters may be modified via the external device 304 and transmitted to the sensing device via the cellular modem 322.

### Ionic Sensors

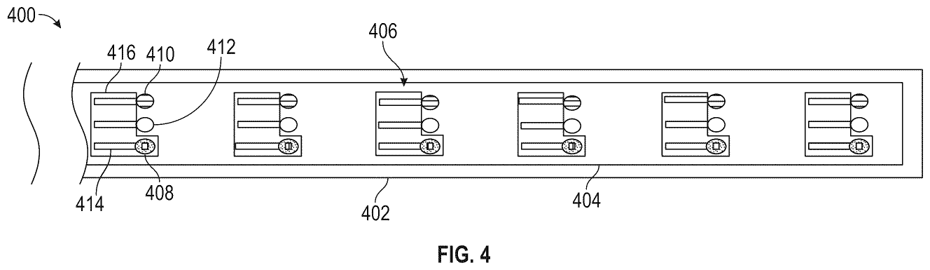

FIG. 4 shows an exemplary ionic sensor assembly 400 of the sensing device, in accordance with some aspects. The ionic sensor assembly 400 may be positioned on an external surface of the housing 402 of the sensing device. The ionic sensor assembly 400 may include a substrate 404 and at least one ionic sensor 406 disposed on said substrate 404. The substrate 404 may be composed of a non-metallic material. For example, the substrate 404 may be a printed circuit having conductive lines therein for electrically coupling the ionic sensor 406 to the controller of the device (e.g., controller 312 shown in FIG. 3 and described above).

The ionic sensor assembly 400 may include at least one ionic sensor 406, such as 1, 2, 3, 4, 5, 6, 7, 8, or more ionic sensors 406, dependent on the desired length of the sensing device. The ionic sensors 406 may be positioned about 5-20 cm, 5-15 cm, or 5-10 cm apart from one another along the substrate 404. For example, the distance between a given pair of ionic sensors 406 may be greater than or equal to 2, 5, 8, 10, 12, or 15 cm. In some examples, the distance between a given pair of ionic sensors 406 may be less than or equal to about 8, 10, 12, 15, 18, 20, or 25 cm.

As described herein, each ionic sensor 406 may comprise a reference electrode 408, a working (e.g., nitrate-measuring) electrode 410, and a counter electrode 412. As explained herein, it is to be understood that the working electrode may be configured to measure an ion different from nitrate in the surrounding medium and is not intended to be limited to this ion. For example, the working electrode 410 may be configured for measuring potassium or phosphate. In some examples, the working electrode 410 may be configured for measuring pH, e.g., by measuring hydrogen ions in the surrounding medium. In some examples, the counter electrode 412 may be configured for measuring oxidation reduction potential (ORP). The electrodes may be positioned relative to one another such that the reference electrode 408 is positioned next to the counter electrode 412, and the working electrode 410 is positioned on the other side of the counter electrode 412. In some examples, the reference electrode 408 and working electrode 410 may be within about 10-20 mm of one another, such as about 10-12 mm, 10-14 mm, 10-16 mm, or about 10-18 mm of one another. In some examples, positioning the reference electrode 408 and working electrode 410 in closer proximity to one another than about 10 mm may result in reference ion pollution at the working electrode 408, which can be undesirable for measuring at the working electrode 408.

Each electrode may include a conductive line electrically connecting the electrode to the controller. The conductive line may be composed of copper. Each electrode may include a gold pad disposed on a portion of the copper line. Each electrode may include a carbon layer 414 disposed above at least the gold pad. The carbon layer 414 may be composed of carbon conductive ink. At least a portion of the carbon layer 414 may be in the form of a line, as shown in FIG. 4. The portion of the carbon layer 414 in the form of a line may have a length between about 5-25 mm, such as about 10-20 mm or about 12-18 mm long. The layering of the copper, gold, and carbon may be significant because copper is known to be reactive when exposed to water/ions, which can cause the copper to generate potentials that change depending on the water/ion combination. To avoid this copper reaction, the carbon layer 414 can be used, as carbon is conductive but also inert.

At least a portion of each of the electrodes may be coated with a coating 416. The coating 416 may comprise silicone or an epoxy. Coating the electrodes can prevent water ingress and stabilize the ionic sensor 406. The line of the carbon layer 414 may terminate with an exposed pad, such as a carbon pad, that is not coated with the coating 416. The carbon pad may be in the form of a circle or rectangle having a length, width, or diameter (as applicable) between about 2-8 mm, such as about 3-6 mm or about 4-5 mm.

The working electrode 410 may include one or more layers disposed on the carbon layer. For example, the working electrode 410 can include a polymer-based ion-selective membrane. The ion-selective membrane may be composed such that it is selective to binding with nitrate ions, potassium ions, phosphate ions, or hydrogen ions (e.g., for measuring pH). The ion-selective membrane may include a polymeric matrix such as polyvinyl chloride (PVC) or a similar inert polymer. In the instance the working electrode is configured to bind to nitrate ions, the polymeric matrix may be embedded with a nitrate-specific ionophore that selectively binds to nitrate ions over other ions that may be present in the surrounding medium. The same can be said for other ions—the polymeric matrix can be embedded with a potassium-specific ionophore, a phosphate-specific ionophore, or a hydrogen-specific ionophore that selectively binds to potassium, phosphate, or hydrogen, respectively, over other ions in the surrounding medium. When the desired ions bind to the ionophore, it creates a charge separation across the membrane, resulting in a potential difference, i.e., electrochemical signal, that can be detected by the working electrode 410.

For a working electrode configured for measuring nitrate ions, the ion-selective membrane may include a solute mixture dissolved in a solvent suitable for preparing ion-selective membranes. For example, the ion-selective membrane may include about 0.1-10 g, 0.1-5 g, 0.5-5 g, or 0.8-2 g solute mixture. The solvent may be selected such that it is suitable for the proper dissolution and mixing of the key components such as ionophores, plasticizers, and other additives which may not often be readily soluble in other solvents due to their complex chemical structures. In some examples, the solute mixture may be dissolved in a solution of tetrahydrofuran (THF), cyclohexanone, or the like. The ion-selective membrane may include about 0.1-10 mL, 1-10 mL, 1-5 mL, or 2-4 mL solvent. In the instance the solvent is cyclohexanone, the solute mixture may require a plasticizer, described below, to dissolve the polymer matrix (e.g., PVC).

The solute mixture may include a polymer matrix. For example, the polymer matrix may include polyvinyl chloride (PVC) Polymethyl methacrylate (PMMA), or the like. For example, the solute mixture may include 15-50 wt. %, 20-45 wt. %, or 25-30 wt. % polymer matrix. In some examples, the solute mixture may include greater than or equal to 15, 20, 25, 30, 35, or 40 wt. % polymer matrix. In some examples, the solute may include less than or equal to 25, 30, 35, 40, 45, or 50 wt. % polymer matrix. In the instance the polymer matrix is PMMA, the solute mixture may not include a plasticizer (e.g., o-NPOE or the like).

The solute mixture may include a polymer plasticizer. The polymer plasticizer can improve flexibility and mechanical properties of the ion-selective membrane. Improving the flexibility and mechanical properties of the ion-selective membrane can allow for improved ion exchange and selectively by creating a suitable environment for the ionophore to interact with target ions. In some examples, the polymer plasticizer includes 2-Nitrophenyl octyl ether (o-NPOE), di-n-butyl phthalate (DBP), or the like. In some examples, the solute mixture includes about twice as much polymer plasticizer as polymer matrix. For example, the solute mixture may include polymer plasticizer and polymer matrix in a ratio of about 2:1, 1.5:1, 1.75:1, 2.25:1, or 2.5:1. For example, the solute mixture may include 25-75 wt. %, 50-75 wt. %, or 60-75 wt. % polymer plasticizer. In some examples, the solute mixture may include greater than or equal to 25, 30, 40, 50, 55, 60, or 65 wt. % polymer plasticizer. In some examples, the solute mixture may include less than or equal to 50, 55, 60, 65, 70, or 75 wt. % polymer plasticizer.

The solute mixture may include a nitrate ionophore for selectively binding to nitrate ions within the membrane. With the nitrate ionophore, the membrane can respond specifically to the presence of nitrate ions in a solution. Thus, the nitrate ionophore can act as the key component for detecting nitrate concentrations using an ion-selective electrode. In some examples, the nitrate ionophore can include tridodecylmethyl ammonium nitrate (TDMAN), or tetraoctylammonium bromide (TOA-BR), a combination thereof, or the like. For example, the solute mixture may include 1-10 wt. %, 2-8 wt. %, or 3-5 wt. % nitrate ionophore. In some examples, the solute mixture includes greater than or equal to about 0.1, 0.5, 1, 2, 3, 4, 5, or 8 wt. % nitrate ionophore. In some examples, the solute mixture includes less than or equal to about 2, 3, 4, 5, 6, 7, 8, 9, or 10 wt. % nitrate ionophore.

The solute mixture may include a lipophilic salt for reducing membrane resistance. Reducing the membrane resistance can improve the signal quality and selectivity of the ion-selective electrode by facilitating ion exchange within the membrane without interfering with the primary ionophore's selectivity towards the target ion. In some examples, the solute mixture may include Tetrakis(4-chlorophenyl) borate tetradodecylammonium salt (ETH500), Tetrabutylammonium tetrakis(4-chlorophenyl) borate, or the like. For example, the solute mixture may include 0.1-5 wt. %, 0.2-2 wt. %, or 0.5-1 wt. % lipophilic salt. In some examples, the solute mixture includes greater than or equal to 0.01, 0.05, 0.1, 0.2, 0.3, 0.4, 0.5, 0.6, 0.8, or 1 wt. % lipophilic salt. In some examples, the solute mixture includes less than or equal to 0.5, 0.8, 1, 2, 3, 4, or 5 wt. % lipophilic salt. In some examples, the solute mixture does not include lipophilic salt (e.g., ETH500 or the like).

An exemplary ion-selective matrix composition for detecting nitrate ions is shown below in Table 1. In some examples, the below composition may be provided without ETH500.

| | |
|---|---|
| Component | Amount |

| | | |
|---|---|---|
| Solute Mixture | 1 | g |
| 2-Nitrophenyl octyl ether (o-NPOE) | 64 | wt. % |
| Tetrakis(4-chlorophenyl) borate | 0.5 | wt. % |
| tetradodecylammonium salt (ETH500) | | |
| Polyvinyl Chloride (PVC) | 32.5 | wt. % |
| Tridodecylmethyl ammonium nitrate (TDMAN) | 3 | wt. % |

Solution

| | | |
|---|---|---|
| Tetrahyrofuran (THF) | 3 | mL |

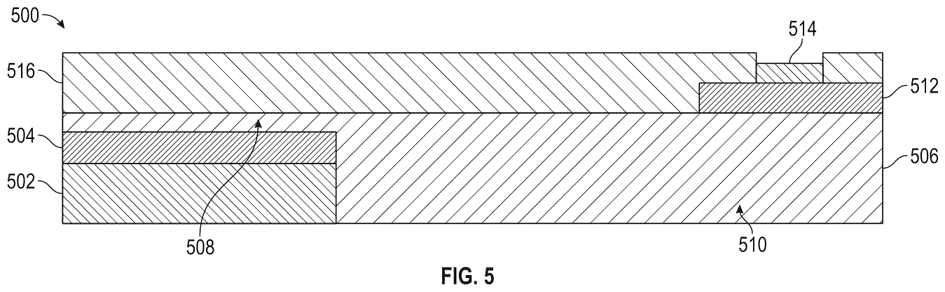

FIG. 5 shows a diagram of an exemplary reference electrode 500 of an ionic sensor, in accordance with some aspects. The reference electrode 500 can include a copper layer 502. The copper layer may be part of a conductive line within the substrate (the PCB). The reference electrode 500 can include a gold layer 504 disposed on the copper layer 502. The gold layer 504 can be a gold pad, as described above with reference to FIG. 4.

The reference electrode 500 can include a carbon layer 506 disposed at least on the gold layer 504. As noted above, the carbon layer 506 may be composed of carbon conductive ink. The line of the carbon layer 506 may be in the form of a carbon line, as described above. As noted above, the carbon layer 506 may have a length between about 5-20 mm, such as about 12-18 mm. Thus, the carbon layer 506 can include a first portion 508 disposed on the gold layer 504 and a second portion 510 adjacent to the first portion 508. The second portion 510 of the carbon layer 506 may flank the copper layer 502 and/or the gold layer 504, as shown in FIG. 5. The second portion 510 of the carbon layer 506 may include a pad of carbon, as noted above, having a length, width, or diameter (as applicable) between about 2-10 mm.

The reference electrode 500 may differ from the working and counter electrodes described herein in that the reference electrode 500 can include a silver layer 512 disposed on the carbon layer 506. Particularly, the silver layer 512 may be disposed on the second portion 510 of the carbon layer 506. The silver layer 512 may be composed of a silver conductive ink. The silver conductive ink may include pure silver. The silver layer 512 may be in the form of a pad having a length, width, or diameter (dependent on the shape of the pad) between about 2-10 mm, such as about 3-8 mm or about 2-6 mm.

At least a portion of the silver layer 512 may be electroplated with iodine, shown in FIG. 5 as silver iodide layer 514. The electrodeposition process is described in greater detail below with reference to an exemplary method of making the reference electrode. The silver iodide layer 514 may be in the form of a pad having a length, width, or diameter (dependent on the desired shape of the pad) between about 1-5 mm, such as about 2-4 mm. The thickness of the silver iodide layer 514 may be selected such that the desired release profile of the iodine over time is achieved. Thus, the thickness of the silver iodide layer 514 may influence the lifespan of the reference electrode 500.

As noted above, the reference electrode 500 can include a coating 516. The coating 516 may coat all but the silver iodide layer 514, as shown in FIG. 5. The coating 516 may be a passivation layer composed of silicone, epoxy resin, or another similar material.

The method for making the reference electrode 500 may be as follows. On a substrate (e.g., a PCB), a conductive (e.g., copper) line may be created. An end of the copper line (502) may be plated with a gold pad. A line of carbon can be printed on the gold pad and can terminate with a carbon pad. The assembly can be cured in an oven for about an hour at about 120° C. In some examples, the assembly can be cured for about 30-90 minutes, about 45-90 minutes, or about 60-60 minutes. In some examples, the assembly can be cured at a temperature of about 100-150° C., about 100-125° C., or about 120-150° C.

After the first curing, the carbon pad can be coated with a layer of silver and cured again. The assembly can be cured in an oven for about an hour at about 120° C. In some examples, the assembly can be cured for about 30-90 minutes, about 45-90 minutes, or about 60-60 minutes. In some examples, the assembly can be cured at a temperature of about 100-150° C., about 100-125° C., or about 120-150° C.

After the second curing, the assembly can be coated, leaving only a portion of the silver pad exposed. In some examples, the iodine may be electrodeposited prior to coating the assembly. In either case, the assembly can then be placed in a bath having free iodine (I⁻) ions available. This electrodeposition solution can contain about 0.1 M sodium iodide, such as about 0.01-1 M, 0.05-0.5 M, or about 0.08-0.2 M sodium iodide. The sodium iodide solution may be produced by mixing sodium iodide with deionized water. A power supply can be connected to the electrochemical cell and can apply a current (e.g., a cathodic current) of about 0.5 mA/cm² for about 60 minutes that causes the silver layer to be electrodeposited with silver iodide. In some examples, the amplitude of the current may be between about 0.01-5 mA/cm², 0.1-2 mA/cm², or about 0.5-1 mA/cm². The current can be applied to the electrochemical cell for about 30-90 minutes, about 45-90 minutes, or about 60-75 minutes.

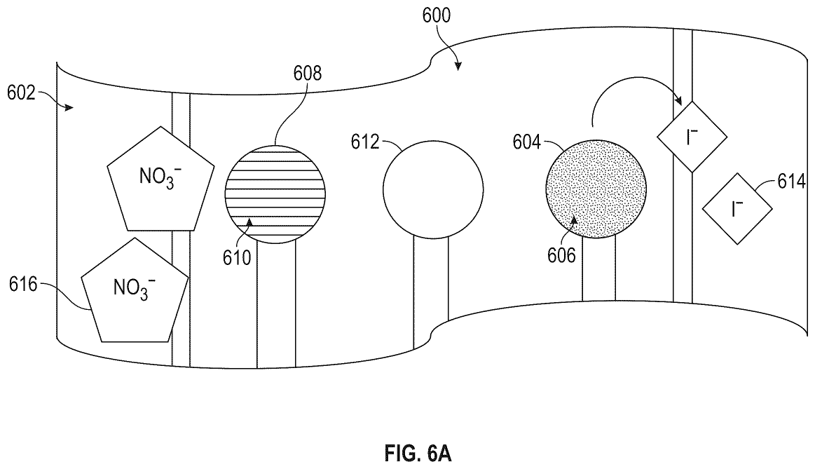

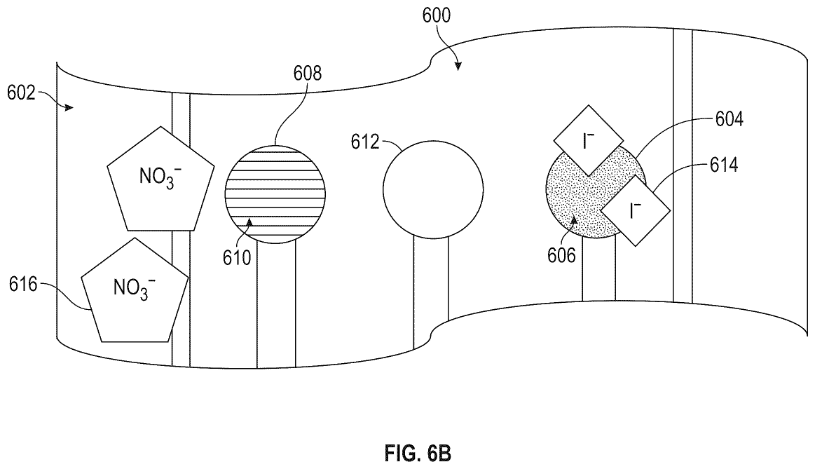

As noted above, the reference electrode 500 can release iodine ions to the surrounding medium that generates a reference potential for measuring an ion concentration, such as nitrate, potassium, or phosphate at the working electrode. In some examples, the reference electrode generates a reference potential for measuring pH, e.g., by detecting hydrogen ions at the working electrode. FIG. 6A shows an exemplary diagram of iodine ion release from the reference electrode 604 and subsequent nitrate detection at the nitrate-measuring electrode 608; and FIG. 6B shows an exemplary diagram of iodine ion recapture at the reference electrode 604, in accordance with some aspects. In FIG. 6A, the device 600 is inserted into a surrounding medium 602, such as soil.

As will be described in greater detail below with reference to FIG. 7, to activate the reference electrode 604, a current pulse can be sent through the counter electrode 612 and the reference electrode 604. The current pulse (e.g., a cathodic current pulse) can cause the release of iodine ions 614 from the silver-iodide coating 606 of the reference electrode 604. The iodine ion release can create a reference potential against which an ion (e.g., nitrate, phosphate, potassium, or hydrogen) concentration in the surrounding medium 602 can be measured. As stated otherwise herein, it is to be understood that other ions may be measured in accordance with the methods and devices described herein and said methods and devices are not intended to be limited to nitrate, phosphate, potassium, and hydrogen. After the current pulse, the electrochemical signal between the potential at the reference electrode 604 and the nitrate-measuring electrode 608 can be measured. The potential difference can vary based on the concentration of nitrate ions in the sample. The electrochemical signal measured at the nitrate-measuring electrode 608 can be correlated to the concentration of nitrate ions 616 in the surrounding medium 602 because of the ion-selective membrane 610 coated on the nitrate-measuring electrode 608 that interacts with the nitrate ions 616.

The counter electrode 612 can balance the release of iodine ions from the reference electrode 614 during the pulsing process. The counter electrode 612 can additionally detect electrochemical signals representative of the oxidation reduction potential of the surrounding medium 602. The counter electrode 612 may conduct this measurement following the initial current pulse. This ORP data can promote the natural nitrogen cycle in the soil.

Following measurements by the nitrate-measuring electrode 608 and the counter electrode 612, a current pulse (e.g., an anodic current pulse) can be applied through the reference electrode 604 and counter electrode 612 that can cause recapture of the iodine ions 614. In essence, the pulsing of the current can generate an infinite reference electrode that prevents significant leaching of the ions from the reference electrode over time.

FIG. 7 shows a diagram of an exemplary activation circuit 700 for driving an ionic sensor assembly, in accordance with some aspects. It is to be understood that components of the circuit diagram 700 may derive from one or more parts of the sensing device. In some examples, the activation circuit 700 is part of a controller, for example, controller 312 shown and described herein with respect to FIG. 3. The circuit diagram 700 can include a voltage source 702, which may be the voltage source described herein with respect to FIG. 3 or may be communicatively coupled to said voltage source. The voltage source 702 can power the activation circuit 700. The activation circuit 700 can include at least one current source 706, 708 coupled to said voltage sources 702, 704. The current sources 706, 708 may be selectively activated and deactivated to cause release and recapture of ions from the reference electrode. For example, the current sources 706, 708 may be a transistor used as a switch to open and close the circuit. As the voltage source 702 is in series with the current source 706, the activation circuit 700 may include a second voltage source 704 for powering the activation circuit 700 when the current source 706 is deactivated. In this way, current can be drawn in both directions. The second voltage source 704 can operate at ½ voltage, as shown in FIG. 7, such that recovering current occurs at a voltage higher than ½ V and releasing current occurs at a voltage lower than ½ V to reverse the current. Also, by artificially raising the sensor ‘resting’ voltage to ½ V, providing a +V and −V power supply to the amplifier can be avoided.

The activation circuit 700 may include an amplifier 710 that can increase the amplitude of the signal at the amplifier 710 prior to releasing it to the surrounding medium at its output (signified as Vout). The amplifier 710 can be electrically coupled to each of the current sources 706, 708.

The activation circuit 700 can include at least one multiplexer 712, 714 coupled to the ionic sensor(s) of the sensing device. For example, the activation circuit 700 can include a multiplexer 712 coupled to one or more reference electrodes 716 and one or more working (e.g., nitrate-measuring) electrodes 718. Other ion-measuring electrodes are also possible in this arrangement. The activation circuit 700 can include a multiplexer 714 coupled to the one or more reference electrodes 716 and one or more counter (e.g., ORP-measuring) electrodes 720. The multiplexers 712, 714 may multiplex, or selectively activate and deactivate, the electrodes. The operating parameters of the multiplexer 712, 714 may be set using respective “SEL” inputs, as shown in FIG. 7.

Operation of the activation circuit 700 to pulse current through the reference electrodes 716 can be described as follows. The multiplexer 712 can be set to R1 (i.e., reference electrode 1) and the multiplexer 714 can be set to O1 (i.e., counter electrode 1). The current source 708 can be turned on to generate a reverse current between the R1 and O1 electrodes. The R1 electrode can release iodine ions in accordance with the magnitude and duration selected. For example, the activation circuit 700 may be configured such that the current source 708 sends a pulse of reverse current having a magnitude of about 1-25 μA, 1-10 μA, 2-8 μA, or about 4-6 μA. The current pulse may have a length between about 0.1-10 s, such as about 0.2-8 s, 0.5-5 s, or about 1-2 s.

Immediately after delivering the current pulse, the multiplexer 712 can be set to W1 (i.e., working electrode 1), the multiplexer 714 can be set to R1, and the electrochemical signal at the working electrode can be measured. Following ion measurement, the oxidation-reduction potential (ORP) can be measured. To do so, the multiplexer 712 can be set to R1, the multiplexer 714 can be set to O1, and the electrochemical signal at the counter electrode can be measured. It may be crucial to measure at substantially the same time every time, so the methods described herein may require such precise measuring.

Following ion (and, optionally ORP measurements), the multiplexer 712 can be set to R1, the multiplexer 714 can be set to O1, and the current source 706 can be turned on to generate a forward current between the R1 and 1 electrodes. The R1 electrode can then recapture the iodine ions in accordance with the magnitude and duration selected. The forward current may have substantially the same magnitude and/or duration as the reverse current. For example, the forward current may have a magnitude of about 1-25 μA, 1-10 μA, 2-8 μA, or about 4-6 μA. The current pulse may have a length between about 0.1-10 s, such as about 0.2-8 s, 0.5-5 s, or about 1-2 s.

This current pulsing method can be repeated for each of the remaining ionic sensors of the sensing device. As described herein, the controller coupled to the ionic sensor assembly may selectively activate the ionic sensors such that only one ionic sensor is performing measurements at a time.

### Moisture Sensors

FIG. 8 shows a diagram of an exemplary probe 800 for sensing moisture of the surrounding medium, in accordance with some aspects. The probe 800 can be part of the sensing device 100 described above. As noted above, the probe 800 can be contained within a chamber of the housing 802 of the sensing device.

The probe 800 can include a moisture sensing assembly 804 disposed on a substrate 806. The substrate 806 may be a printed circuit board (PCB). The substrate 806 may comprise a non-metallic material and conductive lines extending between components connected thereto, as discussed in greater detail below. The moisture sensing assembly can include at least one moisture sensor 808, such as 1, 2, 3, 4, 5, 6, 7, 8, or more moisture sensors 808, dependent on the desired length of the probe 800. The moisture sensors 808 may be positioned along the substrate 806 about 5-20 cm, 5-15 cm, or 5-10 cm apart from one another. For example, the distance between a given pair of moisture sensors 808 may be greater than or equal to 2, 5, 8, 10, 12, or 15 cm. In some examples, the distance between a given pair of moisture sensors 808 may be less than or equal to about 8, 10, 12, 15, 18, 20, or 25 cm.

A given moisture sensor 808 may include one or more electrical contacts 810 and one or more antennas 812 surrounding the one or more electrical contacts 810. For example, the moisture sensor 808 may include a pair of electrical contacts 810 and a pair of antennas 812 surrounding the electrical contacts 810 (respectively). One antenna of the pair of antennas 812 can serve as the active element and the other antenna can serve as the ground element. The antennas 812 may be a capacitive sensor that measures the reactance of the surrounding medium, which can be correlated to the moisture content of the surrounding medium. The one or more antennas 812 may be composed of a thin brass material and may take the shape of a circular or semi-circular arc, or ring. For example, a single antenna 812 may comprise two semi-circular arcs, which can reduce the distance a sensed capacitance must travel to the corresponding electrical contact 810. Reducing the distance by which the sensed capacitance signal must travel can reduce impedance and potential signal noise.

The moisture sensor 808 may electrically connect to the substrate 806 at the one or more electrical contacts 810. In some examples, the electrical contact 810 shorts the opposing sides of the antenna 812 to prevent unwanted noise from electromotive force (EMF).

The probe 800 can include a controller 814 (e.g., as described above with respect to FIG. 3 as moisture controller 312) individually electrically connected to each of the moisture sensors 808 of the moisture sensing assembly 804 to drive the moisture sensors 808. For example, the substrate 806 may comprise one or more conductive lines extending between each electrical contact 810 and the controller 814. Additionally or alternatively, the probe 800 may comprise insulated wires extending between each electrical contact 810 and the controller 814. The controller 814 may selectively control the moisture sensors 808 such that one moisture sensor 808 is activated at a time. In some examples, the moisture sensors 808 are activated sequentially, in a repeated fashion. Activating one moisture sensor 808 at a time can simplify the circuitry necessary to control the moisture sensing assembly 804.

The controller 814 may operate the moisture sensors 808 at a frequency between about 1-500 MHz. For example, the controller 814 may operate the moisture sensors 808 at a frequency between about 100-500 MHz, 150-400 MHz, or 200-300 MHz. In some examples, the controller 814 may operate the moisture sensors 808 at a frequency of greater than or equal to about 1 MHz, 50 MHz, 100 MHz, 150 MHz, 200 MHz, 250 MHz, or 300 MHz. In some examples, the controller 814 may operate the moisture sensors 808 at a frequency of less than or equal to about 200 MHz, 250 MHz, 300 MHz, 350 MHz, 400 MHz, 450 MHz, or 500 MHz. The frequency may be selected such that the calibration of the moisture sensors 808 is predictable and thus reliable.

The controller 814 may be communicatively coupled to a second controller 816 disposed on the substrate 806 for transmitting electrical signals measured by the moisture sensing assembly 804 that are representative of the moisture content of the surrounding medium. For example, the controller 814 may comprises a transceiver for transmitting the measured data to the controller 816. In some examples, the controller 814 is communicatively coupled to the controller 816 by one or more conductive lines extending within or along the substrate 806. In some examples, the probe 800 comprises just one controller. In some examples, the controller 814 is positioned at a distal portion of the substrate 806, whereas the controller 816 is positioned at a proximal portion of the substrate 806, proximate to the telemetry components. Alternative arrangements of the controller(s) are also possible, as will be appreciated by one of ordinary skill in the art.

The controller 816 can electrically connect to the ionic sensor assembly of the sensing device at a connector 818. The connector 818 may comprise one or more sockets for receiving one or more pins extending from the substrate of the ionic sensor assembly, or vice versa (the connector 818 may include pins connectable to respective sockets on the substrate of the ionic sensor assembly). The housing 802 can include necessary openings for facilitating connection between an externally placed ionic sensor assembly and an internal controller 816.

With brief reference to FIG. 4, the controller 816 may be individually electrically connected to each of the ionic sensors 406 of the ionic sensor assembly to drive the ionic sensors 406. For example, the substrate 404 may include one or more conductive lines extending between the controller 816 and each electrode of the ionic sensors 406. As described with respect to FIG. 7, the controller 816 may selectively control the ionic sensors 406 such that only one ionic sensor 406 is activated at a time. In some examples, the ionic sensors 406 are activated sequentially, in a repeated fashion. Activating one ionic sensor 406 at a time can simplify the circuitry necessary to control the ionic sensing assembly 400.

Returning to FIG. 8, the controller (e.g., controller 816 if there is more than one controller, otherwise controller 814) may be communicatively coupled to the telemetry components of the sensing device via a wired connection 820. In some examples, the controller may be communicatively coupled to the central controller of the sensing device via a wireless connection. The controller 816 can include a transceiver for transmitting measured data to the central controller.

The probe 800 can include one or more thermistors 822 for measuring temperature changes of the moisture sensing assembly 804. For example, each moisture sensor 808 may be equipped with a thermistor 822. The thermistor 822 may be disposed between the antennas 812 of a given moisture sensor 808, as shown in FIG. 8. Each thermistor 822 may be coupled to a reference circuit (e.g., of controller 814) for detecting a change in resistance at the thermistor 822. A change in temperature of the moisture sensor 808 can correspond to a change in resistance.

In some examples, the interstitial space within the chamber of the housing 802 surrounding the probe 800 may be filled with an insulating material, such as foam. The foam can add rigidity to the probe 800 and allow for thinner walls of the housing 802, which in turn can improve the sensitivity of the moisture sensing assembly 804. The foam material may additionally or alternatively prevent heat transfer within the chamber of the housing 802. For example, in the context of soil, the part of the probe 800 closer to the surface may experience varying temperature that, without an insulating material, could transfer throughout the chamber and affect the accuracy of signal detection at the moisture sensors 808.

### EMBODIMENTS

The following embodiments are exemplary and are not intended to limit the scope of the disclosure provided herein.

Embodiment 1. A sensing device for measuring nitrate concentration of a surrounding medium, the sensing device comprising:
at least one current source;
at least one ionic sensor comprising:
   a reference electrode configured to release and recapture ions based on current pulses received from the current source to generate a reference potential;
   a counter electrode configured to balance the release of ions from the reference electrode; and
   a nitrate-measuring electrode configured to detect electrochemical signals representative of the nitrate concentration of the surrounding medium based on the reference potential; and
at least one controller electrically connected to the at least one ionic sensor to receive the electrochemical signals from the nitrate-measuring electrode and determine the nitrate concentration of the surrounding medium based on the electrochemical signals.

Embodiment 2. The sensing device of embodiment 1, wherein the counter electrode is configured to detect electrochemical signals representative of the oxidation reduction potential (ORP) of the surrounding medium based on the reference potential.

Embodiment 3. The sensing device of embodiment 2, wherein the at least one controller is configured to receive the electrochemical signals from the counter electrode to determine the ORP of the surrounding medium based on the electrochemical signals.

Embodiment 4. The sensing device of any one of embodiments 1-3, comprising at least one moisture sensor positioned proximate to the at least one ionic sensor array and configured to detect electrical signals representative of a moisture content of the surrounding medium.

Embodiment 5. The sensing device of embodiment 4, wherein the at least one controller is electrically connected to the at least one moisture sensor to receive the electrical signals from the at least one moisture sensor and determine the moisture content of the surrounding medium.

Embodiment 6. The sensing device of any one of embodiments 1-5, wherein the at least one controller comprises at least one multiplexer configured to selectively activate and deactivate the reference electrode, the counter electrode, and the nitrate-measuring electrode to cause the release and recapture of the ions at the reference electrode, and to enable the nitrate-measuring electrode to detect electrochemical signals representative of the nitrate concentration of the surrounding medium.

Embodiment 7. The sensing device of any one of embodiments 1-6, wherein the surrounding medium is soil.

Embodiment 8. The sensing device of any one of embodiments 1-7, wherein the at least one ionic sensor is disposed on a first substrate.

Embodiment 9. The sensing device of embodiment 8, wherein the first substrate attaches to a housing of the sensing device containing the at least one controller such that the at least one ionic sensor and the at least one controller are electrically connected.

Embodiment 10. The sensing device of any one of embodiments 4-9, wherein the at least one moisture sensor is disposed on a second substrate.

Embodiment 11. The sensing device of embodiment 10, wherein the at least one controller is disposed on the second substrate.

Embodiment 12. The sensing device of any one of embodiments 4-11, wherein the at least one controller comprises a first controller configured to determine the moisture content and a second controller configured to determine at least the nitrate concentration.

Embodiment 13. The sensing device of embodiment 12, wherein the first controller is electrically connected to the second controller to receive moisture content data from the second controller.

Embodiment 14. The sensing device of any one of embodiments 1-13, wherein the reference electrode comprises:
a copper layer;
a gold layer disposed on the copper layer;
a carbon layer comprising a first portion disposed on the gold layer and a second portion adjacent to the first portion; and
a silver layer disposed on the second portion of the carbon layer;
a coating disposed on the first portion of the carbon layer and the silver layer such that a portion of the silver layer is exposed; and
a silver iodide layer electrodeposited in the exposed portion of the silver layer.

Embodiment 15. The sensing device of any one of embodiments 1-14, wherein the counter electrode comprises a carbon electrode.

Embodiment 16. The sensing device of any one of embodiments 1-15, wherein the nitrate-measuring electrode comprises a carbon layer and an ion-selective membrane disposed on the carbon layer.

Embodiment 17. The sensing device of embodiment 16, wherein the ion-selective membrane comprises:
a solute mixture comprising:
   25-75 wt. % of 2-Nitrophenyl octyl ether (o-NPOE) or di-n-butyl phthalate (DBP);
   15-50 wt. % of polyvinyl chloride (PVC) or polymethyl methacrylate (PMMA); and
   1-10 wt. % of Tridodecylmethyl ammonium nitrate (TDMAN) and/or tetraoctylammonium bromide; and
a solution comprising tetrahydrofuran (THF) or cyclohexanone.

Embodiment 18. The sensing device of embodiment 17, wherein the solute mixture comprises 0.1-5 wt. % of Tetrakis(4-chlorophenyl) borate tetradodecylammonium salt (ETH500) or tetrabutylammonium tetrakis(4-chlorophenyl) borate.

Embodiment 19. The sensing device of embodiment 17 or 18, wherein the ion-selective membrane comprises between 0.1 g and 5 g of the solute mixture and between 1 mL and 10 mL of the solution.

Embodiment 20. The sensing device of embodiment 17 or 19, wherein the ion-selective membrane comprises:
a solute mixture comprising:
   25-75 wt. % of 2-Nitrophenyl octyl ether (o-NPOE);
   15-50 wt. % of polyvinyl chloride (PVC); and
   1-10 wt. % of Tridodecylmethyl ammonium nitrate (TDMAN); and
a solution comprising tetrahydrofuran (THF).

Embodiment 21. The sensing device of embodiment 20, wherein the solute mixture comprises 0.1-5 wt. % of Tetrakis(4-chlorophenyl) borate tetradodecylammonium salt (ETH500).

Embodiment 22. The sensing device of any one of embodiments 1-21, comprising at least one cellular modem coupled to the at least one controller and configured to send at least nitrate concentration data to an external device.

Embodiment 23. The sensing device of any one of embodiments 1-22, wherein the at least one ionic sensor comprises a plurality of ionic sensors, and the at least one controller is configured to selectively activate each ionic sensor such that one ionic sensor measures at a time.

Embodiment 24. A method for measuring nitrate concentration of soil, comprising:
generating a current pulse between a reference electrode and a counter electrode of a sensing device inserted into soil, whereby the first pulse of current causes release of ions from the reference electrode that generates a reference potential; and
following the current pulse and based on the reference potential, measuring electrochemical signals representative of nitrate concentration of the soil between the reference electrode and a nitrate-measuring electrode of the sensing device.

Embodiment 25. The method of embodiment 24, comprising, following measuring the electrochemical signals representative of nitrate concentration of the soil between the reference electrode and the nitrate-measuring electrode of the sensing device, measuring electrochemical signals representative of oxidation reduction potential (ORP) of the soil between the reference electrode and the nitrate-measuring electrode by the counter electrode.

Embodiment 26. The method of embodiment 24 or 25, comprising, following measuring the nitrate concentration, generating a second current pulse between the reference electrode and the counter electrode that causes recapture of the ions onto the reference electrode.

Embodiment 27. The method of embodiment 26, wherein the first current pulse is a cathodic current and the second current pulse is an anodic current.

Embodiment 28. The method of any one of embodiments 24-27, comprising activating, by at least one multiplexer, the reference electrode and the counter electrode to emit the current pulse between the reference electrode and the counter electrode.

Embodiment 29. The method of embodiment 28, comprising:
deactivating, by the at least one multiplexer, the reference electrode and the counter electrode; and
activating, by the at least one multiplexer, the nitrate-measuring electrode and the reference electrode to enable measurement of the electrochemical signals between the reference electrode and the nitrate-measuring electrode.

Embodiment 30. The method of any one of embodiments 24-29, wherein the ions comprise iodine ions.

Embodiment 31. The method of any one of embodiments 24-30, comprising measuring electrical signals representative of a moisture content of the soil by a moisture sensor proximate to the reference electrode, the nitrate-measuring electrode, and the counter electrode.

Embodiment 32. The method of any one of embodiments 24-31, wherein the current pulse has a magnitude between 1 μA and 25 μA.

Embodiment 33. The method of any one of embodiments 24-32, wherein the current pulse has a pulse width between 0.1 s and 5 s.

Embodiment 34. A reference electrode comprising:
a copper layer;
a gold layer disposed on the copper layer;
a carbon layer comprising a first portion disposed on the gold layer and a second portion adjacent to the first portion; and
a silver layer disposed on the second portion of the carbon layer;
a coating disposed on the first portion of the carbon layer and the silver layer such that a portion of the silver layer is exposed; and
a silver iodide layer electrodeposited in the exposed portion of the silver layer.

Embodiment 35. The reference electrode of embodiment 34, wherein the exposed portion of the silver layer has a length and/or a width between 1.5-4.5 mm.

Embodiment 36. The reference electrode of embodiment 34 or 35, wherein the silver layer has a length and/or a width between 2-6 mm.

Embodiment 37. The reference electrode of any one of embodiments 34-36, wherein the silver layer comprises pure silver.

Embodiment 38. The reference electrode of any one of embodiments 34-37, wherein the carbon layer has a length of about 10-20 mm.

Embodiment 39. The reference electrode of any one of embodiments 34-38, wherein the coating comprises at least one of silicone and epoxy resin.

### EXAMPLES

The following examples are merely illustrative and are not intended to limit the scope of the disclosure provided herein.

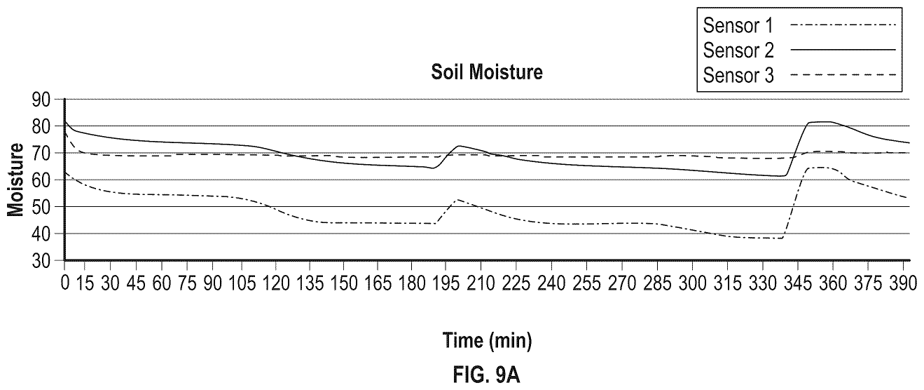

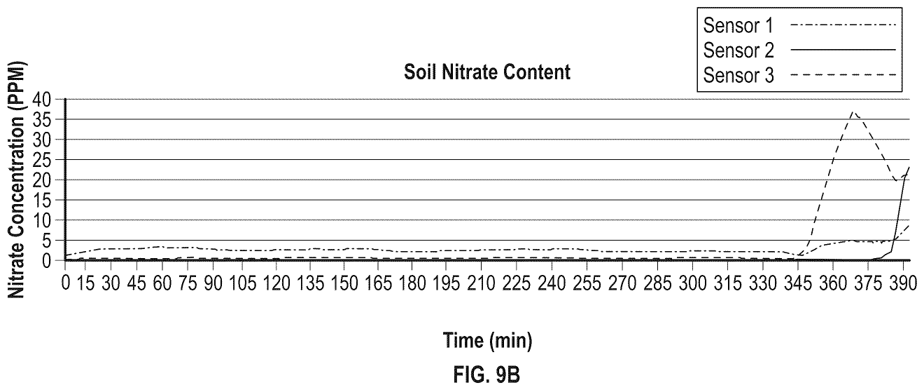

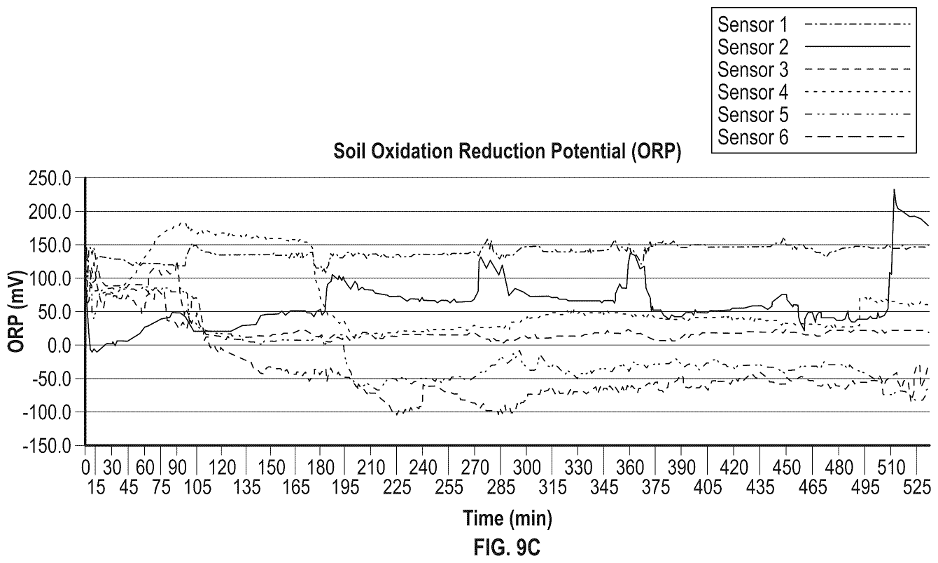

As described herein, the sensing device can measure moisture content of a surrounding medium. FIGS. 9A-9C show measured properties of soil using an exemplary sensing device described in accordance with the embodiments provided herein. The data shown in FIGS. 9A-9C was measured over the same duration of about 6-7 hours, using the same sensing device. In each graph shown in FIGS. 9A-9C, the data was plotted in increments of 15 minutes along the x-axis. The graphs depict data measured using 3 different sensors along the same sensing device. In particular, sensor 1 is 10 cm from the surface, sensor 2 is 20 cm from the surface, and sensor 3 is 30 cm from the surface. So, sensor 1 would capture a change based on irrigation/rain first, then sensor 2, and then sensor 3 (possibly).

FIG. 9A depicts measured moisture content of the soil. In the graph shown in FIG. 9A, moisture content is depicted on a scale of 0-100, where 0 is air and 100 is immersed in water. FIG. 9B depicts measured nitrate concentration in the soil. In the graph shown in FIG. 9B, nitrate concentration is measured in parts per million (PPM). FIGS. 9A-9B prove that moisture is the carrier of the nitrate in the soil. FIG. 9C depicts measured oxidation reduction potential (ORP) in the soil. In the graph shown in FIG. 9C, ORP is measured in mV. If the ORP of the soil is below 50-100 mV, the soil may be denitrifying. If the ORP of the soil is above about 100 mV, the soil may be capable of generating nitrate. In all, the graphs shown in FIGS. 9A-9C depict proper functioning of the sensing device for measuring nitrate concentration, moisture, and ORP of soil.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

For the purpose of clarity and a concise description, features are described herein as part of the same or separate examples; however, it will be appreciated that the scope of the disclosure includes examples having combinations of all or some of the features described.

What is claimed is:

1. A sensing device for measuring nitrate concentration of a surrounding medium, the sensing device comprising:
at least one current source;
at least one ionic sensor comprising:
a reference electrode configured to release and recapture ions based on current pulses received from the current source to generate a reference potential;
a counter electrode configured to balance the release of ions from the reference electrode; and
a nitrate-measuring electrode configured to detect electrochemical signals representative of the nitrate concentration of the surrounding medium based on the reference potential; and
at least one controller electrically connected to the at least one ionic sensor to receive the electrochemical signals from the nitrate-measuring electrode and determine the nitrate concentration of the surrounding medium based on the electrochemical signals.

2. The sensing device of claim 1, wherein the counter electrode is configured to detect electrochemical signals representative of the oxidation reduction potential (ORP) of the surrounding medium based on the reference potential.

3. The sensing device of claim 2, wherein the at least one controller is configured to receive the electrochemical signals from the counter electrode to determine the ORP of the surrounding medium based on the electrochemical signals.

4. The sensing device of claim 1, comprising at least one moisture sensor positioned proximate to the at least one ionic sensor array and configured to detect electrical signals representative of a moisture content of the surrounding medium.

5. The sensing device of claim 4, wherein the at least one controller is electrically connected to the at least one moisture sensor to receive the electrical signals from the at least one moisture sensor and determine the moisture content of the surrounding medium.

6. The sensing device of claim 1, wherein the at least one controller comprises at least one multiplexer configured to selectively activate and deactivate the reference electrode, the counter electrode, and the nitrate-measuring electrode to cause the release and recapture of the ions at the reference electrode, and to enable the nitrate-measuring electrode to detect electrochemical signals representative of the nitrate concentration of the surrounding medium.

7. The sensing device of claim 1, wherein the surrounding medium is soil.

8. The sensing device of claim 1, wherein the at least one ionic sensor is disposed on a first substrate.

9. The sensing device of claim 8, wherein the first substrate attaches to a housing of the sensing device containing the at least one controller such that the at least one ionic sensor and the at least one controller are electrically connected.

10. The sensing device of claim 4, wherein the at least one moisture sensor is disposed on a second substrate.

11. The sensing device of claim 10, wherein the at least one controller is disposed on the second substrate.

12. The sensing device of claim 4, wherein the at least one controller comprises a first controller configured to determine the moisture content and a second controller configured to determine at least the nitrate concentration.

13. The sensing device of claim 12, wherein the first controller is electrically connected to the second controller to receive moisture content data from the second controller.

14. The sensing device of claim 1, wherein the reference electrode comprises:
a copper layer;
a gold layer disposed on the copper layer;
a carbon layer comprising a first portion disposed on the gold layer and a second portion adjacent to the first portion; and
a silver layer disposed on the second portion of the carbon layer;
a coating disposed on the first portion of the carbon layer and the silver layer such that a portion of the silver layer is exposed; and
a silver iodide layer electrodeposited in the exposed portion of the silver layer.

15. The sensing device of claim 1, wherein the counter electrode comprises a carbon electrode.

16. The sensing device of claim 1, wherein the nitrate-measuring electrode comprises a carbon layer and an ion-selective membrane disposed on the carbon layer.

17. The sensing device of claim 16, wherein the ion-selective membrane comprises:
a solute mixture comprising:
25-75 wt. % of 2-Nitrophenyl octyl ether (o-NPOE) or di-n-butyl phthalate (DBP);
15-50 wt. % of polyvinyl chloride (PVC) or polymethyl methacrylate (PMMA); and
1-10 wt. % of Tridodecylmethyl ammonium nitrate (TDMAN) and/or tetraoctylammonium bromide; and
a solution comprising tetrahydrofuran (THF) or cyclohexanone.

18. The sensing device of claim 17, wherein the solute mixture comprises 0.1-5 wt. % of Tetrakis(4-chlorophenyl) borate tetradodecylammonium salt (ETH500) or tetrabutylammonium tetrakis(4-chlorophenyl) borate.

19. The sensing device of claim 17, wherein the ion-selective membrane comprises between 0.1 g and 5 g of the solute mixture and between 1 mL and 10 mL of the solution.

20. The sensing device of claim 17, wherein the ion-selective membrane comprises:
a solute mixture comprising:
25-75 wt. % of 2-Nitrophenyl octyl ether (o-NPOE);
15-50 wt. % of polyvinyl chloride (PVC); and
1-10 wt. % of Tridodecylmethyl ammonium nitrate (TDMAN); and
a solution comprising tetrahydrofuran (THF).

21. The sensing device of claim 20, wherein the solute mixture comprises 0.1-5 wt. % of Tetrakis(4-chlorophenyl) borate tetradodecylammonium salt (ETH500).

22. The sensing device of claim 1, comprising at least one cellular modem coupled to the at least one controller and configured to send at least nitrate concentration data to an external device.

23. The sensing device of claim 1, wherein the at least one ionic sensor comprises a plurality of ionic sensors, and the at least one controller is configured to selectively activate each ionic sensor such that one ionic sensor measures at a time.

24. A method for measuring nitrate concentration of soil, comprising:
generating a current pulse between a reference electrode and a counter electrode of a sensing device inserted into soil, whereby the first pulse of current causes release of ions from the reference electrode that generates a reference potential; and
following the current pulse and based on the reference potential, measuring electrochemical signals representative of nitrate concentration of the soil between the reference electrode and a nitrate-measuring electrode of the sensing device.

25. The method of claim 24, comprising, following measuring the electrochemical signals representative of nitrate concentration of the soil between the reference electrode and the nitrate-measuring electrode of the sensing device, measuring electrochemical signals representative of oxidation reduction potential (ORP) of the soil between the reference electrode and the nitrate-measuring electrode by the counter electrode.

26. The method of claim 24, comprising, following measuring the nitrate concentration, generating a second current pulse between the reference electrode and the counter electrode that causes recapture of the ions onto the reference electrode.

27. The method of claim 26, wherein the first current pulse is a cathodic current and the second current pulse is an anodic current.

28. The method of claim 24, comprising activating, by at least one multiplexer, the reference electrode and the counter electrode to emit the current pulse between the reference electrode and the counter electrode.

29. The method of claim 28, comprising:
deactivating, by the at least one multiplexer, the reference electrode and the counter electrode; and
activating, by the at least one multiplexer, the nitrate-measuring electrode and the reference electrode to enable measurement of the electrochemical signals between the reference electrode and the nitrate-measuring electrode.

30. The method of claim 24, wherein the ions comprise iodine ions.

31. The method of claim 24, comprising measuring electrical signals representative of a moisture content of the soil by a moisture sensor proximate to the reference electrode, the nitrate-measuring electrode, and the counter electrode.

32. The method of claim 24, wherein the current pulse has a magnitude between 1 μA and 25 μA.

33. The method of claim 24, wherein the current pulse has a pulse width between 0.1 s and 5 s.

34. A reference electrode comprising:
a copper layer;
a gold layer disposed on the copper layer;
a carbon layer comprising a first portion disposed on the gold layer and a second portion adjacent to the first portion; and
a silver layer disposed on the second portion of the carbon layer;
a coating disposed on the first portion of the carbon layer and the silver layer such that a portion of the silver layer is exposed; and
a silver iodide layer electrodeposited in the exposed portion of the silver layer.

35. The reference electrode of claim 34, wherein the exposed portion of the silver layer has a length and/or a width between 1.5-4.5 mm.

36. The reference electrode of claim 34, wherein the silver layer has a length and/or a width between 2-6 mm.

37. The reference electrode of claim 34, wherein the silver layer comprises pure silver.

38. The reference electrode of claim 34, wherein the carbon layer has a length of about 10-20 mm.

39. The reference electrode of claim 34, wherein the coating comprises at least one of silicone and epoxy resin.
